(19) United States
(12) Patent Application Publication
(10) Pub. No.: US 2023/0263671 A1
(43) Pub. Date: Aug. 24, 2023

(54) SAFETY MEDICAL SWABS

(71) Applicant: EMED LABS, LLC, Miami, FL (US)

(72) Inventors: Jeffrey M. Schumm, Miami, CA (US); Michael W. Ferro, JR., Palm Beach, FL (US); Michael J. Mina, Miami, FL (US); James Thomas Heising, Richland, WA (US)

(21) Appl. No.: 18/172,712

(22) Filed: Feb. 22, 2023

Related U.S. Application Data
(60) Provisional application No. 63/367,962, filed on Jul. 8, 2022.
(60) Provisional application No. 63/313,055, filed on Feb. 23, 2022.

(51) Int. Cl. A61F 13/38; A61B 10/00

(52) U.S. Cl. CPC A61F 13/38; A61B 10/0051; A61F 2013/422

(57) ABSTRACT

A medical swab for medical diagnostic testing in provided herein. In particular, the medical swab may include a crumple zone configured to crumple, elastically deform, plastically deform, bend, flex, collapse, and/or give when an axial force and/or a radial force is applied to the medical swab. In some embodiments, a stem of the medical swab may retract into or extend from one or more bases. In some embodiments, the medical swab can include a telescoping mechanism configured to control retraction and/or extension of the stem. In some embodiments, the medical swab can include a color changing tip and/or a color changing stem.

### PRIORITY APPLICATIONS

This application claims priority to U.S. Provisional Application No. 63/313,055, filed Feb. 23, 2022, and U.S. Provisional Application No. 63/367,962, filed Jul. 8, 2022, each of which are incorporated herein by reference. Any and all applications for which a foreign or domestic priority claim is identified in the Application Data Sheet as filed with the present application are hereby incorporated by reference under 37 CFR 1.57.

### BACKGROUND

### Field

The present application is directed to a swab, which may be used in testing, such as diagnostic medical testing.

### Description

Use of telehealth to deliver healthcare services has grown consistently over the last several decades and has experienced very rapid growth in the last several years. Telehealth can include the distribution of health-related services and information via electronic information and telecommunication technologies. Telehealth can allow for long-distance patient and health provider contact, care, advice, reminders, education, intervention, monitoring, and remote admissions. Often, telehealth can involve the use of a user or patient’s personal user device, such as a smartphone, tablet laptop, personal computer, or other device. For example, a user or patient can interact with a remotely located medical care provider using live video, audio, or text-based chat through the personal user device. Generally, such communication occurs over a network, such as a cellular or internet network.

Remote or at-home healthcare testing and diagnostics can solve or alleviate some problems associated with in-person testing. For example, health insurance may not be required, travel to a testing site is avoided, and tests can be completed at a testing user’s convenience. However, remote or at-home testing introduces various additional logistical and technical issues, such as guaranteeing timely test delivery to a testing user, providing test delivery from a testing user to an appropriate lab, ensuring adequate user experience, ensuring proper sample collection, ensuring test verification and integrity, providing test result reporting to appropriate authorities and medical providers, and connecting testing users with medical providers who are needed to provide guidance and/or oversight of the testing procedures remotely.

### SUMMARY

For purposes of this summary, certain aspects, advantages, and novel features of the invention are described herein. It is to be understood that not all such advantages necessarily may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein.

In a first aspect, a medical swab for diagnostic testing can include a stem comprising a crumple zone, and a tip coupled to the stem, wherein the crumple zone comprises a portion of the stem configured to deform when a force larger than a predetermined threshold is applied to the tip.

The medical swab can include one or more of the following features in any combination: (a) wherein the stem comprises a first material, and the crumple zone comprises a second material; (b) wherein the stem comprises a first cross sectional area, and the crumple zone comprises a second cross sectional area, wherein the first cross sectional area is larger than the second cross sectional area; (c) wherein the crumple zone comprises an entire stem; (d) wherein the stem comprises a distal end and a proximal end, and wherein the crumple zone is centered along a length of the stem between the distal end of the stem and the proximal end of the stem; (e) wherein the stem comprises a distal end and a proximal end, and wherein the crumple zone comprises a distal end and a proximal end, and wherein a first distance between the distal end of the crumple zone and the distal end of the stem is smaller than a second distance between the proximal end of the crumple zone and a proximal end of the stem; (f) wherein the stem comprises a distal end and a proximal end, and wherein the crumple zone comprises a distal end and a proximal end, and wherein a first distance between the distal end of the crumple zone and the distal end of the stem is larger than a second distance between the proximal end of the crumple zone and a proximal end of the stem; (g) a base, wherein the base comprises an opening configured to receive a distal end of the stem; (h) wherein the stem is configured to retract into the base and extend out of the base between a retracted position and an extended position; (g) wherein the tip is configured to change from a first color to a second color when the tip is exposed to a use indicator; and/or other features as described herein.

In another aspect, a medical swab for diagnostic testing can include a stem comprising a distal end and a proximal end; a tip coupled to the proximal end; and a base comprising a distal end and a proximal end, wherein the proximal end of the base comprises an opening, and wherein the opening of the base is configured to receive the distal end of the stem, and the stem is configured to retract into the base and extend out of the base between a retracted position and an extended position.

The medical swab can include one or more of the following features in any combination: (a) wherein a cross-sectional area of the distal end of the stem is larger than a cross-section area of the opening of the base such that the stem cannot be removed from the base; (b) wherein the base comprises a first portion of a telescoping mechanism and the stem comprises a second portion of the telescoping mechanism, and wherein the telescoping mechanism controls retraction and/or extension of the stem between the retracted position and the extended position; (c) wherein the base is a first base, and the medical swab further comprises a second base configured to receive the first base, and wherein the first base is configured to retract into the second base and extend out of the base between a retracted position and an extended position; (d) wherein the stem comprises a crumple zone; (e) wherein the tip is configured to change from a first color to a second when the tip is exposed to a use indicator; and/or other features as described herein.

In another aspect, a medical swab for diagnostic testing can include a stem comprising a distal end and a proximal end; and a tip coupled to the proximal end, wherein the tip is configured to change from a first color to a second color when the tip is exposed to a use indicator.

The medical swab can include one or more of the following features in any combination: (a) wherein the use indicator can comprise a predetermined temperature threshold, moisture, and/or a positive or negative diagnosis of a medical condition; (b) wherein the stem comprises a crumple zone; (c) wherein the base comprises an opening configured to receive the distal end of the stem; and/or other features as described herein.

### BRIEF DESCRIPTION OF THE DRAWINGS

These and other features, aspects, and advantages of the present application are described with reference to drawings of certain embodiments, which are intended to illustrate, but not limit the present disclosure. It is to be understood that the attached drawings are for the purpose of illustrating concepts disclosed in the present application and may not be to scale.

FIGS. 1A-1C illustrate an example medical swab including a crumple zone according to some embodiments described herein.

FIGS. 2A and 2B illustrate an example medical swab that includes one or more bases according to some embodiments described herein.

FIGS. 3-5 illustrate example medical swabs that include a telescoping mechanism according to some embodiments described herein.

FIGS. 6 and 7 show an example medical swab that includes both a crumple zone and a telescoping mechanism according to some embodiments described herein.

FIGS. 8A and 8B illustrate example color changing medical swabs according to some embodiments described herein.

### DETAILED DESCRIPTION

Although several embodiments, examples and illustrations are disclosed below, it will be understood by those of ordinary skill in the art that inventions described herein extend beyond the specifically disclosed embodiments, examples, and illustrations and includes other uses of the inventions and obvious modifications and equivalents thereof. Embodiments of the inventions are described with reference to the accompanying figures, wherein like numerals refer to the like elements throughout. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

In some embodiments, a medical swab for sample collecting can include a crumple zone built into the stem. In some embodiments, the crumple zone can be configured to collapse when an axial or radial force threshold is reached. The force threshold may be less than a force that causes tissue damage.

In some embodiments, the swab can be used during sample collection. For example, during sample collection (e.g., a sample from the back of the throat as in a strep test), it may be difficult to see and/or feel where the tip of the swab is located and/or what the tip of the swab is touching. In some embodiments, it may be possible to push too hard on parts of the sample area (e.g., the throat or other tissues) with the swab. In some embodiments, to prevent damage to cells and tissue, the crumple zone on the stem may limit the amount of force that may be applied by the swab.

In some embodiments, the specific force threshold of the crumple zone may be selected depending on the type of sampling or the body location where the swab will be used. For example, a nose swab crumple zone may have a higher threshold than a throat swab crumple zone.

In some embodiments, the crumple zone may limit force applied to the swab in an axial direction and/or a radial direction. For example, when doing a swirling motion of a swab tip within the nasal cavity, the crumple zone may flex (e.g., elastically or plastically deform) if the swab is pushed radially against the nose with more than a threshold amount of force.

In some embodiments, the crumple zone can be a different material than the other parts of the stem or can be a thinner material compared to other parts of the stem.

In some embodiments, the entire stem can act as a force limiting crumple zone where the stem is a uniform material configured to flex or bend when a threshold axial and/or radial force is reached.

In some embodiments, a medical swab for sample collecting can include a telescoping stem. In some embodiments, at-home diagnostic test kits include several components, and the sample collection swab is generally the longest component. In some embodiments, the length of the swab can be a limiting factor in being able to reduce package size of the diagnostic test box. In some embodiments, reducing the package size can be beneficial for increasing the number of boxes that can be shipped in a truck shipment, increasing the number of boxes that can be accommodated on a store shelf, decreasing the amount of space the test occupies in a traveler’s luggage, minimizing shipping damage (e.g., crushed boxes) to the box, etc.

In some embodiments, a swab with a telescoping stem can be used to reduce the length of the swab so that packaging size can be reduced without compromising the quality of the test components or the testing experience.

In some embodiments, the swab tip can be coupled with a swab stem at a first end. In some embodiments, the second end of the swab stem that is opposite the swab tip can be inserted into a swab base. In some embodiments, a most compact position a large portion/majority/entirety of the stem can be nested within the base.

In some embodiments, before use, a user can extend (e.g., pull, twist, push, etc.) the swab stem relative to the base so that the overall length of the swab increases. In some embodiments, the length of the swab can increase through an intermediate length to a maximally extended length. In some embodiments, the maximally extended length can correspond with a standard swab length (e.g., 3 inches, 5 inches, 6 inches, etc.).

In some embodiments, depending on the selected lengths of the stem and the base, the compact length may be between 50-80% of the length of extended length. In some embodiments, other ranges are possible depending on specific design.

In some embodiments, additional segments can be added to further minimize the compact length. For example, a second base can be included wherein the first base and the stem both nest within the second base and all three components can be made shorter without reducing the fully extended length.

In some embodiments, a telescoping mechanism can allow for relative movement between the swab segments (e.g., between the stem and the base). In some embodiments, the telescoping mechanism can be one of various mechanisms including a leadscrew thread, one or more detents, and/or a friction fit engaged by a tapered geometry.

In some embodiments, the swab can include a crumple zone and telescoping features. In some embodiments, the swab can provide improved safety (e.g., does not apply axial and/or radial forces greater than a selected threshold) and fit within small packaging constraints.

In some embodiments, the swab can be a single use, disposable item formed from a paper/cardboard, plastic, and/or elastomer materials. In some embodiments, the swab components can be pre-assembled and can be shipped and stored in a compact configuration.

In some embodiments, the swab base can be a reusable piece in which a disposable swab is inserted. In some embodiments, the inserted swab can be a standard medical swab as used today or can be a swab with a crumple zone. In some embodiments, if the base is a reusable base, the opening in which the swab stem is inserted may be elastic or otherwise flexible/conformable to hold the swab stem securely. In some embodiments, the end of the inserted stem can be prevented from traveling too far/all the way into the base by a surface or other feature that blocks its axial movement.

In some embodiments, a portion of the swab may change colors based on a change in temperature. The swab used in a medical diagnostic test may initially appear white in color. The swab may change colors (e.g., blue, yellow, green, black, brown, red, etc.) to indicate information to the user. The information indicated to the user may inform the user that it may be okay to proceed with the medical diagnostic test or that it the user may need to take additional steps before proceeding with the medical diagnostic test. For example, an additional step may include obtaining an additional swab to be used in the medical diagnostic test. In some embodiments, the swab may change colors to indicate to the user that the swab may have obtained a sufficient sample from the user. For example, a sufficient sample may have been obtained if the swab reached a sufficient depth within an orifice of the user to collect a sample to be analyzed during the administration of the medical diagnostic test. In some embodiments, upon the swab reaching a sufficient depth within the user’s orifice, the swab may change colors to indicate so.

In some embodiments, for example, the swab can change colors to indicate to the user that the swab has been used or is no longer sterile. In some embodiments, the swab can provide notice to the user that the user should obtain another swab before proceeding with the medical diagnostic test. In some embodiments, the swab can include a moisture sensitive material. In some embodiments, the size of the swab can vary. For example, the size of the swab used in a medical diagnostic test for a child can be smaller than the size of the swab used in a medical diagnostic test for an adult. Additionally, the shape of the swab used in a medical diagnostic test can vary. The shape of the swab can be defined by the orifice in which it can be inserted into. For example, the shape of the swab can be circular, cylindrical, conical, or other various shapes. This can provide a greater range of medical diagnostic tests that can be administered.

Tailoring tools used during a medical diagnostic test or conveying information to a user regarding the adequacies of the administration of the medical diagnostic test can be important. Accordingly, it can be beneficial to provide the user with a swab that can vary in shape and change in color based on a change in temperature. This can allow the user to obtain more accurate results from the medical diagnostic test. For example, in some cases, the color of the swab can change upon moisture absorption.

In some embodiments, a user can obtain a swab that can be used during administration of a medical diagnostic test. In some embodiments, the swab can be sterile and unused. In some embodiments, the swab can appear white in color. In some embodiments, upon use of the swab during administration of a medical diagnostic test, the swab can alter its appearance in color to indicate information to the user.

In some embodiments, the swab can appear a color other than white to indicate information to the user. In some embodiments, the information can include sufficient sample collection, type of sample collected, etc.

### Medical Swabs

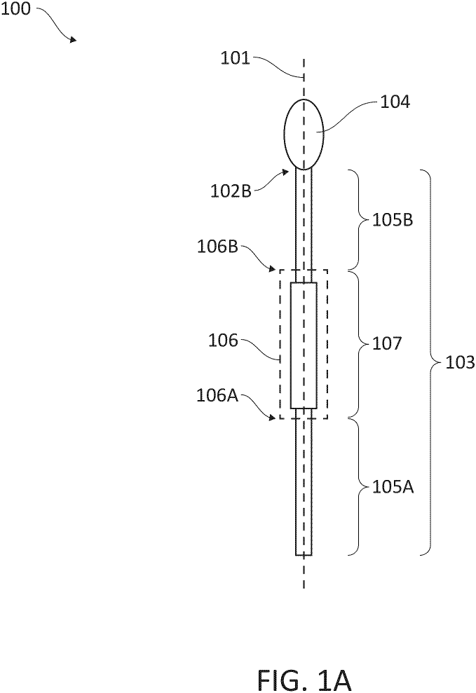

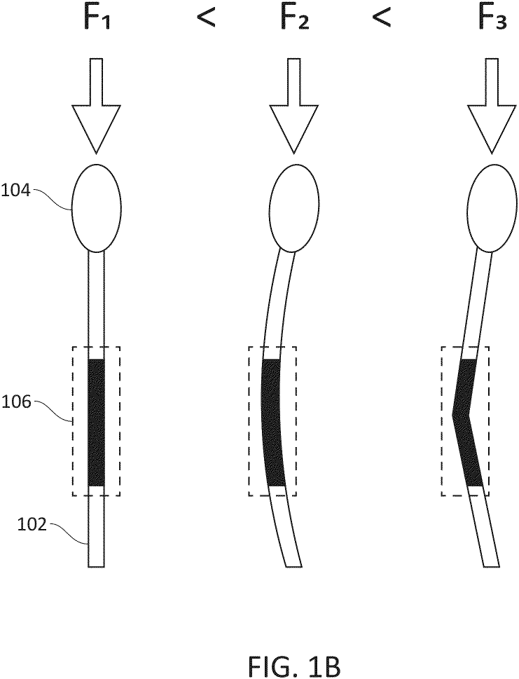

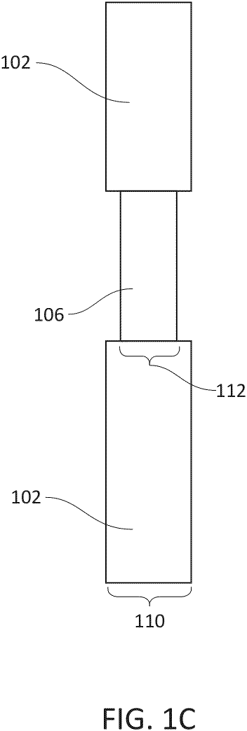

FIG. 1A illustrates an example of a medical swab 100 for sample collection. In some embodiments, the medical swab 100 can be used at a location of a user’s body. In some embodiments, the medical swab 100 can be inserted in a nose of the user, an ear of the user, a mouth of the user, a throat of the user, a cut or incision on the body of the user, and/or any other location of the user’s body. In some embodiments, the medical swab 100 can include a stem 102 and a tip 104. In some embodiments, the stem 102 can include a distal end 102A and a proximal end 102B. In some embodiments, the tip 104 can be coupled to the proximal end 102B of the stem 102. In some embodiments, the stem 102 can include nylon, ABS, polystyrene, wood, paper, plastic, and/or any other material. In some embodiments, the tip 104 can include cotton, rayon, polyester, foam, polyurethane, and/or any other material.

In some embodiments, the stem 102 can include a length 103, wherein the length 103 is a distance from the distal end 102A and a proximal end 102B. In some embodiments, an axial force 108A can applied to the medical swab 100, the stem 102, and/or the tip 104, wherein the axial force 108A is a force along an axis 101 extending from the distal end 102A to the proximal end 102B of the stem 102. In some embodiments, a radial force 108B can be applied to the medical swab 100, the stem 102, and/or the tip 104, wherein the radial force 108B is a force perpendicular to the axis 101. In some embodiments, both an axial force 108A and a radial force 108B can be applied to the medical swab 100, the stem 102 and/or the tip 104.

In some embodiments, the stem 102 can include a crumple zone 106. In some embodiments, the crumple zone 106 can be built into the stem 102. In some embodiments, the crumple zone 106 can be a portion of the stem 102 configured to crumple, elastically deform, plastically deform, bend, flex, collapse, and/or give when an axial force 108A and/or a radial force 108B is applied to the medical swab 100, the stem 102, and/or the tip 104. In some embodiments, the entire stem 102 can be the crumple zone 106. In some embodiments, the crumple zone 106 can include a length 107. In some embodiments, the length 107 of the crumple zone 107 can be a portion of the length 103 of the stem 102. In some embodiments, length 107 of the crumple zone 106 can be a percentage of the length 103 of the stem 102. In some embodiments, the length 107 of the crumple zone 106 can be about 10 percent, about 20 percent, about 30 percent, about 40 percent, about 50 percent, about 60 percent, about 70 percent, about 80 percent, about 90 percent, about 100 percent, and/or any value between the aforementioned values, of the length of the stem 102.

In some embodiments, the crumple zone 106 can be positioned along the length 103 of the stem 102. In some embodiments, the crumple zone 106 can be centered along the length 103 of the stem 102 between the distal end 102A of the stem 102 and the proximal end 102B of the stem 102. In some embodiments, the crumple zone 106 can be off-center along the length 103 of the stem 102 between the distal end 102A of the stem 102 and the proximal end 102B of the stem 102.

In some embodiments, a distance 105A between a distal end 106A of the crumple zone 106 and the distal end 102A of the stem 102 can be smaller than a distance 105B between a proximal end 106B of the crumple zone 106 and the proximal end 102B of the stem 102. In some embodiments, the distance 105A between the distal end 106A of the crumple zone 106 and the distal end 102A of the stem 102 can be larger than the distance 105B between the proximal end 106B of the crumple zone 106 and the proximal end 102B of the stem 102. In some embodiments, the distance 105B between the proximal end 106B of the crumple zone 106 and the proximal end 102B of the stem 102 can be smaller than the distance 105A between the distal end 106A of the crumple zone 106 and the distal end 102A of the stem 102. In some embodiments, the distance 105B between the proximal end 106B of the crumple zone 106 and the proximal end 102B of the stem 102 can be larger than the distance 105A between the distal end 106A of the crumple zone 106 and the distal end 102A of the stem 102.

In some embodiments, the crumple zone 106 can include a force threshold. In some embodiments, the crumple zone 106 can be configured to crumple, elastically deform, plastically deform, bend, flex, collapse, and/or give when the axial force 108A and/or the radial force 108B is larger than the force threshold. In some embodiments, if the axial force 108A and/or the radial force 108B is smaller than the force threshold, the crumple zone 106 can be configured to not crumple, elastically deform, plastically deform, bend, flex, collapse, and/or give.

In some embodiments, the crumple zone 106 can include a first force threshold and a second force threshold. In some embodiments, the second force threshold can be larger than the first force threshold. In some embodiments, the crumple zone 106 can be configured to crumple, elastically deform, plastically deform, bend, flex, collapse, and/or give when the axial force 108A and/or the radial force 108B is larger than the first force threshold (i.e., F₂ in FIG. 1B). In some embodiments, the crumple zone 106 can be configured to break and/or snap when the axial force 108A and/or the radial force 108B is larger than the second force threshold (i.e., F₃ in FIG. 1B). In some embodiments, if the axial force 108A and/or the radial force 108B is smaller than the first force threshold, the crumple zone 106 can be configured to not crumple, elastically deform, plastically deform, bend, flex, collapse, and/or give (i.e., F₁ in FIG. 1B).

In some embodiments, the crumple zone 106 can include a different material from the rest of the stem 102. In some embodiments, as shown in FIG. 1C, the stem 102 can include a width 110 and the crumple zone 106 can include a width 112. In some embodiments, the width 112 of the crumple zone 106 can be smaller than the width of the rest of the stem 102. In some embodiments, the crumple zone 106 can be thinner than the rest of the stem 102. In some embodiments, the rest of the stem 102 can be thicker than the crumple zone 106.

In some embodiments, the length 103 of the stem 102, the length 107 of the crumple zone 106, the position of the crumple zone 106 along the length 103 of the stem 102, the force threshold, the first force threshold, and/or the second force threshold can depend on or be based on a type of sample or the location of the user’s body where the swab will be used.

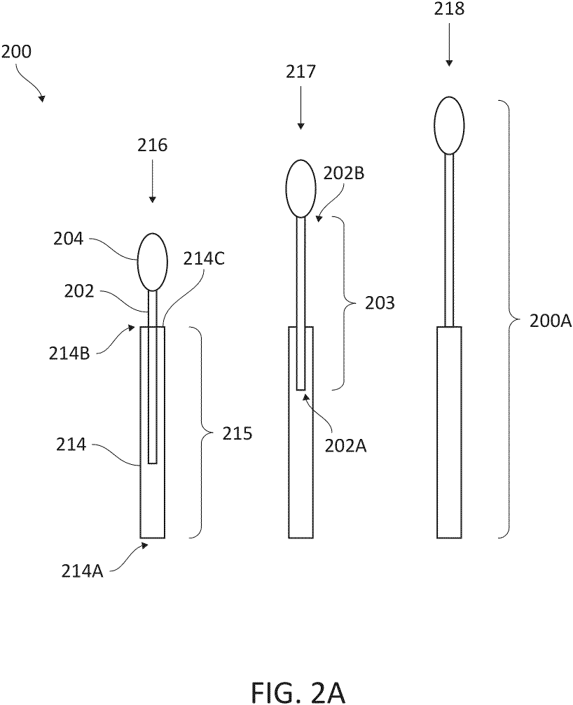

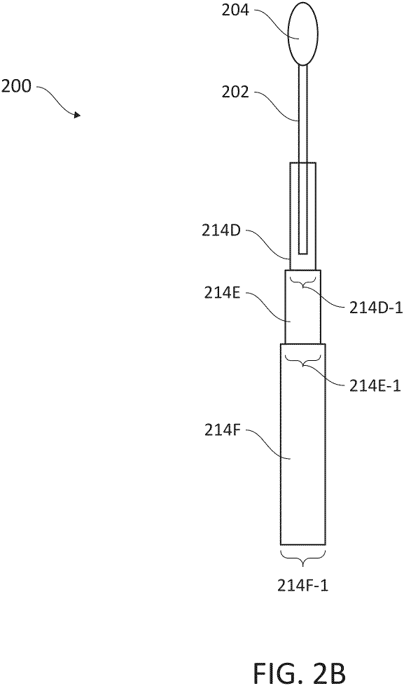

FIG. 2A shows a medical swab 200 for sample collection. In some embodiments, the medical swab 200 can be used at a location of a user’s body. In some embodiments, the medical swab 200 can be inserted in a nose of the user, an ear of the user, a mouth of the user, a throat of the user, a cut or incision on the body of the user, and/or any other location of the user’s body. In some embodiments, the medical swab 200 can include a stem 202 and a tip 204. In some embodiments, the stem 202 can include a distal end 202A and a proximal end 202B. In some embodiments, the stem 202 can include a length 203, wherein the length 203 is a distance from the distal end 202A and a proximal end 202B. In some embodiments, the tip 204 can be coupled to the proximal end 202B of the stem 202. In some embodiments, the stem 102 can include nylon, ABS, polystyrene, wood, paper, plastic, and/or any other material. In some embodiments, the tip 204 can include cotton, rayon, polyester, foam, polyurethane, and/or any other material.

In some embodiments, the stem 202 of the medical swab 200 can be a telescoping stem. In some embodiments, the medical swab 200 can include a base 214. In some embodiments, the base 214 can include a distal end 214A and a proximal end 214B. In some embodiments, the proximal end 214B of the base 214 can include an opening 214C. In some embodiments, the opening 214C can be configured to receive the stem 202 such that the stem 202 can be inserted into the base 214.

In some embodiments, the base 214 can include a length 215. In some embodiments, the length 215 of the base 214 can be a distance from the distal end 214A to the proximal end 214B. In some embodiments, the length 215 of the base 214 can be the same as the length 203 of the stem 202. In some embodiments, the length 215 of the base 214 can be longer than the length 203 of the stem 202. In some embodiments, the length 215 of the base 214 can be shorter than the length 203 of the stem 202. In some embodiments, the length 203 of the stem 202 can be longer than the length 215 of the base 214. In some embodiments, the length 203 of the stem 202 can be shorter than the length 215 of the base 214.

In some embodiments, the stem 202 can be slidably coupled to the base 214. In some embodiments, the stem 202 can be retracted into the base 214 and/or extended from the base 214. In some embodiments, the stem 202 can be removed from the base 214. In some embodiments, the stem 202 cannot be removed from the base 214. In some embodiments, the stem 202 can be retracted and/or extended between a retracted state 216 and an extended state 218. In some embodiments, the stem 202 can be in a partially extended state 217. In some embodiments, when the stem 202 is in the retracted state 216, the distal end 202A of the stem 202 can be positioned at the distal end 214A of the base 214. In some embodiments, when the stem 202 is in the extended state 218, the distal end 202A of the stem 202 can be position at the proximal end 214B of the base. In some embodiments, when the stem 202 is in the partially extended state 218, the distal end 202A of the stem 202 can be positioned between the distal end 214A of the base 214 and the proximal end 214B of the base.

In some embodiments, the medical swab 200 can include a length 200A. In some embodiments, the length 200A of the medical swab 200 can be a distance from the distal end 214A of the base 214 to an end of the tip 204 opposite the proximal end 202B of the stem. In some embodiments, a length 200A of the medical swab 200 when the stem 202 is in the retracted state 216 can be about 10 percent, about 20 percent, about 30 percent, about 40 percent, about 50 percent, about 60 percent, about 70 percent, about 80 percent, and/or any value between the aforementioned values, of the length 200A of the medical swab 200 when the stem is in the extended state 218. In some embodiments, the length 200A of the medical swab 200 when the stem 202 is in the retracted state 216 can be between about 50 percent and about 80 percent of the length 200A of the medical swab 200 when the stem 202 is in the extended state 218.

In some embodiments, as shown in FIG. 2B, the base 214 can be a first base 214D and the medical swab 200 can include a second base 214E. In some embodiments, a width 214D-1 of the first base 214D can be smaller than a width 214E-1 of the second base 214E. In some embodiments, the stem 202 can retract into the first base 214D (i.e., the base 214), and the first base 214D and/or the stem 202 can retract into the second base 214E. In some embodiments, the first base 214D and the second base 214E can form a telescoping base. In some embodiments, as shown in FIG. 2C, the medical swab can include a third base 214F, wherein a width 214F-1 of the third base 214F is larger than the width 214E-1 of the second base 214E. In some embodiments, the stem 202 can retract into the stem 202 can retract into the first base 214D (i.e., the base 214), and the first base 214D and/or the stem 202 can retract into the second base 214E, and the second base 214E, the first base 214D, and/or the stem, can retract into the third base 214F. In some embodiments, the first base 214D, the second base 214E, and the third base 214F can from a telescoping base. In some embodiments, the medical swab 200 can include four bases, five base, six base, and/or more bases, wherein each base retracts into the next base to form a telescoping base.

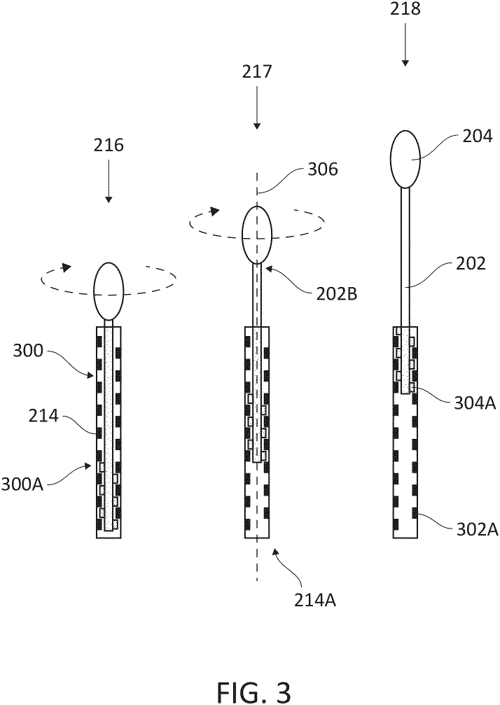

In some embodiments, as shown in FIGS. 3-5 the base 214 can include a telescoping mechanism 300. In some embodiments, as shown in FIG. 3A, the telescoping mechanism 300 can include a leadscrew thread 300A. In some embodiments, the base 214 can include first portion 302A of the leadscrew thread 300A, and the stem 202 can include a second portion 304A of the leadscrew thread 300A. In some embodiments, the second portion 304A can rotatably couple to the first portion 302A such that when the second portion 304A is rotated relative to the first portion 302A, the second portion 304A moves linearly relative to the first portion 302A. In some embodiments, the stem 202 and/or the tip 204 can be rotated around an axis 306 extending from the proximal end 202B of the stem 202 to the distal end 214A of the base 214. In some embodiments, when the stem 202 and/or the tip 204 are rotated in a first direction, the stem 202 can retract into the base 214. In some embodiments, when the stem 202 and/or the tip 204 are rotated in a second direction, the stem 202 can extend from the base 214, as shown in FIG. 3. In some embodiments, the first portion 302A can apply a force to the second portion 304B and/or the second portion 304B can apply a force to the first portion 302A to prevent the stem 202 from retracting and/or extending without the stem 202 and/or the tip 204 being rotated in the first direction or the second direction.

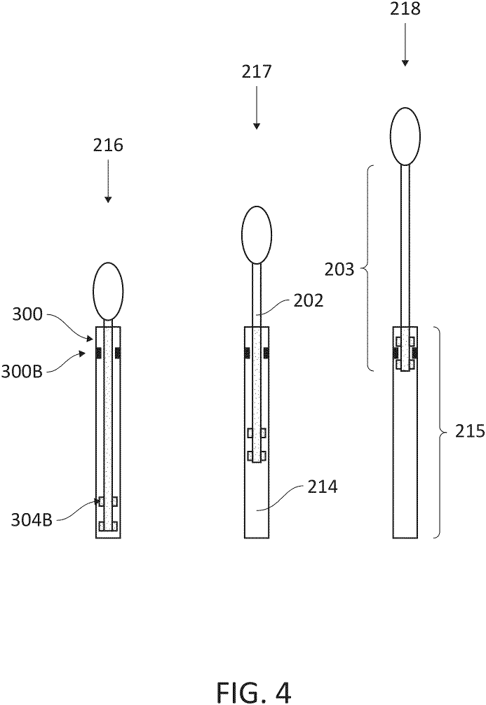

In some embodiments, as shown in FIG. 4, the telescoping mechanism 300 can include one or more detents 300B. In some embodiments, the base 214 can include a first portion 302B of the one or more detents 300B. In some embodiments, the stem 202 can include a second portion 304B of the one or more detents 300B. In some embodiments, when the second portion 304B of the one or more detents 300B is positioned at a location of the first portion 302B of the one or more detents 300B, the first portion 302B apply a force to the second portion 304B to prevent the stem 202 from being retracted into the base 214 or extended from the base 214.

In some embodiments, the first portion 302B of the one or more detents 300B can include one or more holes along the length 215 of the base 214, and the second portion 304B of the one or more detents 300B can include one or more extrusions along the length 203 of the stem 202. In some embodiments, when the one or more extrusions along the length 203 of the stem 202 are positioned at a location of the one or more holes along the length 215 of the base 214, the one or more extrusions can extend into and/or through the one or more holes to prevent the stem 202 from being retracted or extend by the user.

In some embodiments, the first portion 302B of the one or more detents 300B can include one or more extrusions in the base 214, and the second portion 304B of the one or more detents 300B can include one or more extrusions along the length 203 of the stem 202. In some embodiments, when the one or more extrusions along the length 203 of the stem 202 are positioned at a location of the one or more extrusions of the base 214, the one or more extrusions along the length 203 of the stem 202 and the one or more extrusions of the base 214 can be coupled together to prevent the stem 202 from being retracted or extended by the user.

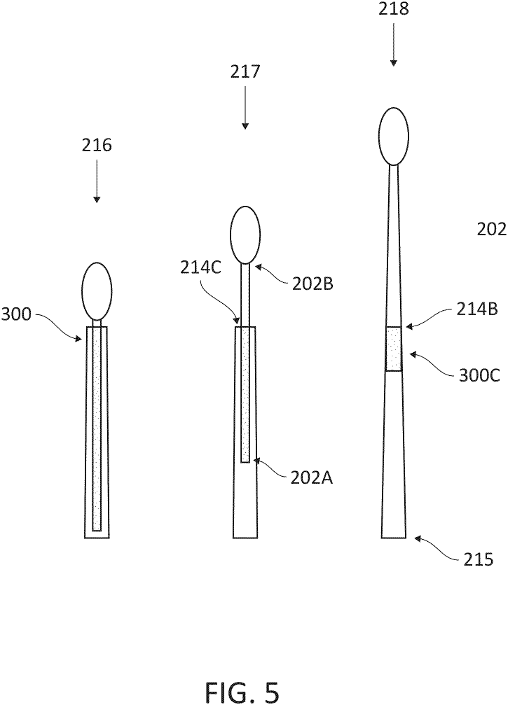

In some embodiments, as shown in FIG. 5 the telescoping mechanism 300 can include a friction fit 300C between the stem 202 and the base 214. In some embodiments, the base 214 can include a tapered geometry. In some embodiments, a cross-sectional area and/or a width of the base 214 at the distal end 214A of the base 214 can be larger than a cross-sectional area and/or a width of the base 214 at the proximal end 214B of the base. In some embodiments, a cross-sectional area and/or a width of the stem 202 at the distal end 202A of the stem 202 can be larger than a cross-sectional area and/or a width of the stem 202 at the proximal end 202B of the stem 202. In some embodiments, the cross-section area and/or the width of the stem 202 at the distal end 202A if the stem 202 can be larger than the cross-sectional area and/or the width of the base 214 at the proximal end 214B of the base 214 In some embodiments, when stem 202 is in the extended state 218 the stem 202 friction between the stem 202 and the base 214 prevent the stem 202 from being removed from the base 214. In some embodiments, the cross-section area and/or the width of the stem 202 at the distal end 202A of the stem 202 can be larger than a cross-sectional area and/or a width of the opening 214C of the base 214 such that when the stem 202 is in the extended state 218, the distal end 202 of the stem cannot fit through the opening 214C.

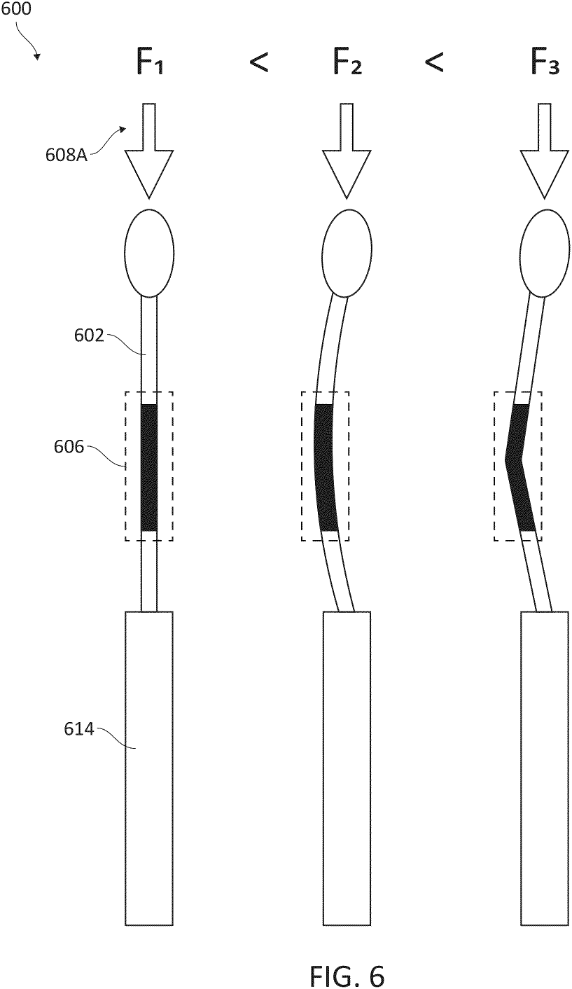

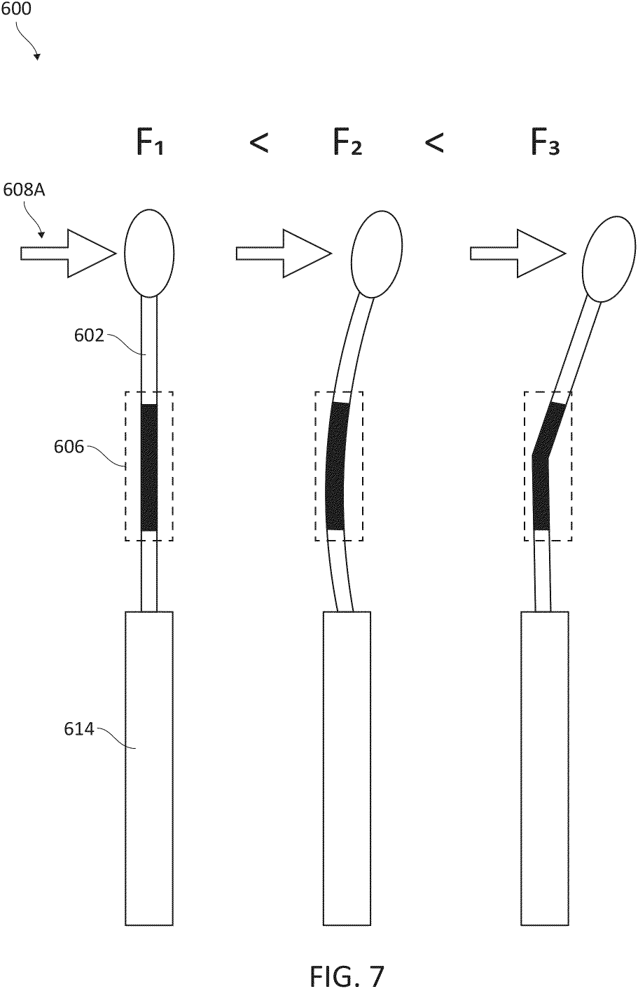

FIGS. 6 and 7 show another medical swab 600 for sample collection. In some embodiments, the medical swab 600 can include a crumple zone 606 and one or more bases 614. In some embodiments, the medical swab 600 can include any features of the medical swab 100, as described with reference to FIGS. 1A-1C, and the medical swab 200, as describe with reference to FIGS. 3-5. In some embodiments, as shown in FIG. 6, the crumple zone 606 can include a force threshold. In some embodiments, the crumple zone 606 can be configured to crumple, elastically deform, plastically deform, bend, flex, collapse, and/or give when an axial force 608A (shown in FIG. 6) and/or the radial force 608B (shown in FIG. 7) is larger than the force threshold. In some embodiments, if the axial force 608A and/or the radial force 608B is smaller than the force threshold, the crumple zone 606 can be configured to not crumple, elastically deform, plastically deform, bend, flex, collapse, and/or give.

In some embodiments, the crumple zone 606 can include a first force threshold and a second force threshold. In some embodiments, the second force threshold can be larger than the first force threshold. In some embodiments, the crumple zone 606 can be configured to crumple, elastically deform, plastically deform, bend, flex, collapse, and/or give when the axial force 608A and/or the radial force 608B is larger than the first force threshold (i.e., F₂ in FIGS. 6 and 7). In some embodiments, the crumple zone 606 can be configured to break and/or snap when the axial force 608A and/or the radial force 608B is larger than the second force threshold (i.e., F₃ in FIGS. 6 and 7). In some embodiments, if the axial force 608A and/or the radial force 608B is smaller than the first force threshold, the crumple zone 606 can be configured to not crumple, elastically deform, plastically deform, bend, flex, collapse, and/or give (i.e., F₁ in FIGS. 6 and 7).

In some embodiments, the medical swab 600 can include the one or more bases 214 of medical swab 200, as described with reference to FIG. 2B. In some embodiments, the medical swab 600 can include the telescoping mechanism 300 as described with reference to FIGS. 3-5. In some embodiments, the medical swab 600 can include the crumple zone 606, the one or more bases 214 of medical swab 200, and the telescoping mechanism 300. In some embodiments, medical swab 600 can provide improved safety, for example, the medical swab 600 does not apply axial and/or radial forces greater than the force threshold and fit within small packaging constraints due to the one or more bases 214 and/or the telescoping mechanism 300.

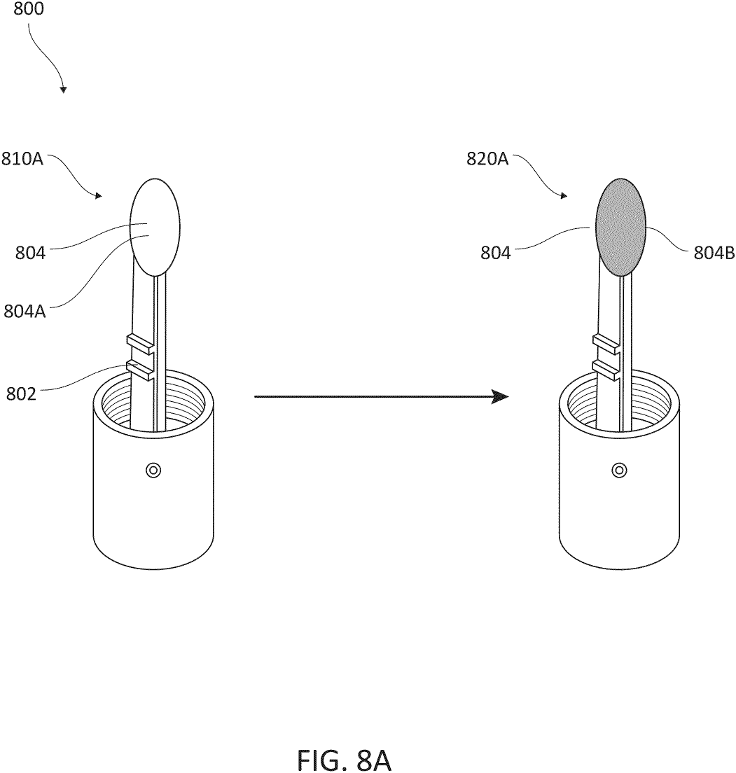

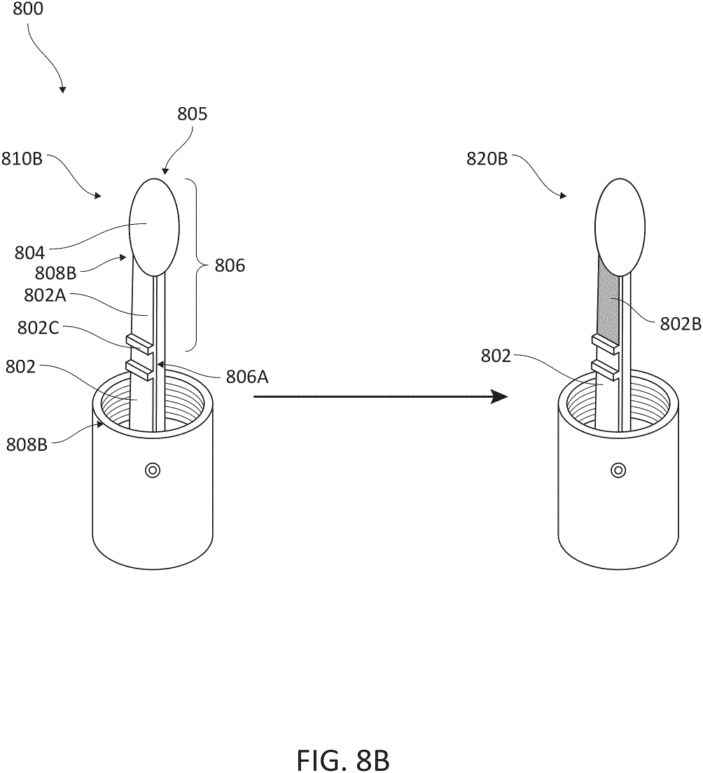

FIGS. 8A-8B is a diagram showing a medical swab 800 configured to change color. As shown in FIG. 8A, in some embodiments, the medical swab 800 can include a tip 804 configured to change color. In some embodiments, the tip 804 of the medical swab 800 can be configured to change color when a user uses the medical swab 800 during administration of a medical diagnostic test and/or after the user uses the medical swab 800 during administration of the medical diagnostic test. In some embodiments, the tip 804 can be configured to change color when the tip 804 is exposed to a use indicator.

In some embodiments, at a first stage 810A, the medical swab 800 can include an unused tip 804A. In some embodiments, the unused tip 804A can appear white in color. In some embodiments, the unused tip 804A can appear as a first color. In some embodiments, the unused tip 804A can be sterile.

In some embodiments, at a second stage 820A, after the user uses the medical swab 800 during administration of the medical diagnostic test, the medical swab 800 can include a used tip 804B. In some embodiments, the used tip 804B can appear non-white. In some embodiments, the used tip 804B can appear as a second color wherein the second color is different from the first color.

In some embodiments, the tip 804 can include temperature sensitive material, and the use indicator can include a predetermined temperature threshold. In some embodiments, the tip 804 can be configured to change color, or transition from an unused tip 804A to a used tip 804B, if the tip 804 is exposed to a temperature above the predetermined threshold. In some embodiments, the predetermined threshold can include a temperature of about 91° F., about 92° F., about 93° F., about 94° F., about 95° F., about 96° F., about 98° F., about 99° F., about 100° F., about 101° F., about 102° F., about 103° F., about 104° F., about 105° F., and/or any value between the aforementioned values.

In some embodiments, the tip 804 can include moisture sensitive material, and the use indicator can include when the tip 804 contacts fluid or gets wet. In some embodiments, the tip 804 can be configured to change color, or transition from an unused tip 804A to a used tip 804B, if the tip 804 contacts fluid or gets wet.

In some embodiments, the tip 804 can be configured for diagnosing or testing various medical conditions (which can be symptomatic conditions or asymptomatic conditions). In some embodiments, the use indicator can include a positive and/or a negative diagnosis for one or more medical conditions. In some embodiments, the tip 804 can be configured to change color, or transition from an unused tip 804A to a used tip 804B, if a user tests positive for a medical condition. In some embodiments, the tip 804 can be configured to change color to a first color if the user tests positive for a first medical condition, and the tip 804 can be configured to change color to a second color if the user tests positive for a second medical condition. In some embodiments, the tip 804 can be configured to not change color or the tip can be configured to change color to a third color if the user tests negative for a medical condition.

In some embodiments, as shown in FIG. 8B, the medical swab 800 can include a stem 802 configured to change color. In some embodiments, the stem 802 of the medical swab 800 can be configured to change color when a user uses the medical swab 800 during administration of a medical diagnostic test and/or after the user uses the medical swab 800 during administration of the medical diagnostic test. In some embodiments, stem 802 can be configured to change color when the stem 802 is exposed to a use indicator.

In some embodiments, at a first stage 810B, the medical swab 800 can include an unused stem 802A. In some embodiments, the unused stem 802A can appear white in color. In some embodiments, the unused stem 802A can appear as a first color. In some embodiments, the unused stem 802A can be sterile.

In some embodiments, at a second stage 820B, after the user uses the medical swab 800 during administration of the medical diagnostic test, the medical swab 800 can include a used stem 802B. In some embodiments, the used stem 802B can appear non-white. In some embodiments, the used stem 802B can appear as a second color wherein the second color is different from the first color.

In some embodiments, the stem 802 can include temperature sensitive material, and the use indicator can include a predetermined temperature threshold. In some embodiments, the stem 802 can be configured to change color, or transition from an unused stem 802A to a used stem 802B, if the stem 802 is exposed to a temperature above a predetermined threshold. In some embodiments, the predetermined threshold can include a temperature of about 91° F., about 92° F., about 93° F., about 94° F., about 95° F., about 96° F., about 98° F., about 99° F., about 100° F., about 101° F., about 102° F., about 103° F., about 104° F., about 105° F., and/or any value between the aforementioned values.

In some embodiments, the stem 802 can include moisture sensitive material, and the use indicator can include when the tip 804 contacts fluid or gets wet. In some embodiments, the stem 802 can be configured to change color, or transition from an unused stem 802A to a used stem 802B, if the stem 802 contacts fluid or gets wet.

In some embodiments, the stem 802 can be configured for diagnosing or testing various medical conditions (which can be symptomatic conditions or asymptomatic conditions). In some embodiments, the use indicator can include a positive and/or a negative diagnosis for one or more medical conditions. In some embodiments, the stem 802 can be configured to change color, or transition from an unused stem 802A to a used stem 802B, if a user tests positive for a medical condition. In some embodiments, the stem 802 can be configured to change color to a first color if the user tests positive for a first medical condition, and the stem 802 can be configured to change color to a second color if the user tests positive for a second medical condition. In some embodiments, the stem 802 can be configured to not change color or the tip can be configured to change color to a third color if the user tests negative for a medical condition.

In some embodiments, the medical swab 800 can be inserted into an orifice of the user’s body, for example, the user’s mouth, the user’s nose, the user’s ear, etc. In some embodiments, the medical swab 800 can be inserted into the orifice to a depth 806, wherein the depth 806 is a distance measured from a proximal end 805 of the tip 804 to location 806A of the stem 802 between a proximal end 808A of the stem 802 and a distal end 808B of the stem 802. In some embodiments, a portion of the stem 802 between the proximal end 808A of the stem 802 and the location 806A of the stem 802 can be configured to change color, or transition from an unused stem 802A to a used stem 802B. In some embodiments, the user, a medical professional, and/or a telehealth proctor can use the portion of the stem 802 between the proximal end 808A of the stem 802 and the location 806A of the stem 802 to determine a size of the orifice of the user’s body. In some embodiments, the user, the medical professional, and/or a telehealth proctor can compare the location 806A to one or more markings 802C on the stem 802 to determine if the medical swab 800 is inserted into the orifice of the user’s body to a depth 806 required for a medical diagnostic test. In some embodiments, if the location 806A is between the one or more markings 802C and the distal end 808B of the stem 802, the user, the medical professional, and/or a telehealth proctor can determine the medical swab 800 was inserted into the orifice of the user’s body to a depth 806 required for the medical diagnostic test. In some embodiments, if the location 806A is between the one or more markings 802C and the proximal end 808A of the stem 802, the user, the medical professional, and/or a telehealth proctor can determine the medical swab 800 was not inserted into the orifice of the user’s body to a depth 806 required for the medical diagnostic test.

In some embodiments, medical swabs 100, 200, and 600 can include any of the features of medical swab 800 as described with reference to FIGS. 8A-8B.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than restrictive sense.

Indeed, although this invention has been disclosed in the context of certain embodiments and examples, it will be understood by those skilled in the art that the invention extends beyond the specifically disclosed embodiments to other alternative embodiments and/or uses of the invention and obvious modifications and equivalents thereof. In addition, while several variations of the embodiments of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the embodiments of the disclosed invention. Any methods disclosed herein need not be performed in the order recited. Thus, it is intended that the scope of the invention herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and subcombinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination. No single feature or group of features is necessary or indispensable to each and every embodiment.

It will also be appreciated that conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open- ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the invention is not to be limited to the particular forms or methods disclosed, but, to the contrary, the invention is to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (e.g., as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein but are to be accorded the widest scope consistent with this disclosure, the principles and the novel features disclosed herein.

What is claimed is:

1. A medical swab for diagnostic testing, comprising:
a stem comprising a crumple zone; and
a tip coupled to the stem,
wherein the crumple zone comprises a portion of the stem configured to deform when a force larger than a predetermined threshold is applied to the tip.

2. The medical swab of claim 1, wherein the stem comprises a first material, and the crumple zone comprises a second material.

3. The medical swab of claim 1, wherein the stem comprises a first cross sectional area, and the crumple zone comprises a second cross sectional area, wherein the first cross sectional area is larger than the second cross sectional area.

4. The medical swab of claim 1, wherein the crumple zone comprises an entire stem.

5. The medical swab of claim 1, wherein the stem comprises a distal end and a proximal end, and wherein the crumple zone is centered along a length of the stem between the distal end of the stem and the proximal end of the stem.

6. The medical swab of claim 1, wherein the stem comprises a distal end and a proximal end, and wherein the crumple zone comprises a distal end and a proximal end, and wherein a first distance between the distal end of the crumple zone and the distal end of the stem is smaller than a second distance between the proximal end of the crumple zone and a proximal end of the stem.

7. The medical swab of claim 1, wherein the stem comprises a distal end and a proximal end, and wherein the crumple zone comprises a distal end and a proximal end, and wherein a first distance between the distal end of the crumple zone and the distal end of the stem is larger than a second distance between the proximal end of the crumple zone and a proximal end of the stem.

8. The medical swab of claim 1, further comprising a base, wherein the base comprises an opening configured to receive a distal end of the stem.

9. The medical swab of claim 8, wherein the stem is configured to retract into the base and extend out of the base between a retracted position and an extended position.

10. The medical swab of claim 1, wherein the tip is configured to change from a first color to a second color when the tip is exposed to a use indicator.

11. A medical swab for diagnostic testing, comprising:
a stem comprising a distal end and a proximal end;
a tip coupled to the proximal end; and
a base comprising a distal end and a proximal end,
wherein the proximal end of the base comprises an opening, and wherein the opening of the base is configured to receive the distal end of the stem, and the stem is configured to retract into the base and extend out of the base between a retracted position and an extended position.

12. The medical swab of claim 11, wherein a cross-sectional area of the distal end of the stem is larger than a cross-section area of the opening of the base such that the stem cannot be removed from the base.

13. The medical swab of claim 11, wherein the base comprises a first portion of a telescoping mechanism and the stem comprises a second portion of the telescoping mechanism, and wherein the telescoping mechanism controls retraction and/or extension of the stem between the retracted position and the extended position.

14. The medical swab of claim 11, wherein the base is a first base, and the medical swab further comprises a second base configured to receive the first base, and wherein the first base is configured to retract into the second base and extend out of the base between a retracted position and an extended position.

15. The medical swab of claim 11, wherein the stem comprises a crumple zone.

16. The medical swab of claim 11, wherein the tip is configured to change from a first color to a second when the tip is exposed to a use indicator.

17. A medical swab for diagnostic testing, comprising:
a stem comprising a distal end and a proximal end; and
a tip coupled to the proximal end,
wherein the tip is configured to change from a first color to a second color when the tip is exposed to a use indicator.

18. The medical swab of claim 17, wherein the use indicator can comprise a predetermined temperature threshold, moisture, and/or a positive or negative diagnosis of a medical condition.

19. The medical swab of claim 17, wherein the stem comprises a crumple zone.

20. The medical swab of claim 17 further comprising a base, wherein the base comprises an opening configured to receive the distal end of the stem.
